(19) United States
(12) Patent Application Publication
(10) Pub. No.: US 2007/0184030 A1
(43) Pub. Date: Aug. 9, 2007

(54) Large scale storage of viable somatic stem and/or progenitor cells

(75) Inventor: Marc Waeterschoot, Schelle (BE)

Correspondence Address: KNOBBE MARTENS OLSON & BEAR LLP, 2040 MAIN STREET, FOURTEENTH FLOOR, IRVINE, CA, 92614, US

(73) Assignee: CRYO-SAVE AG (FORMERLY LIFE SCIENCES AG), CH-8808 PFAFFIKON SWITZERLAND (CH)

(21) Appl. No.: 10/591,409

(22) PCT Filed: Feb. 28, 2005
(86) PCT No.: PCT/EP05/02094

§ 371 Date: Sep. 1, 2006

Related U.S. Application Data
(60) Provisional application No. 60/549,581, filed on Mar. 3, 2004.

(51) Int. Cl. A61K 35/14; C12N 5/08

(52) U.S. Cl. 424/93.7; 435/325; 435/366; 435/372

(57) ABSTRACT

A large scale storage system of viable somatic stem and/or progenitor cells for use in a method of treatment of a disease or a disorder of a patient is described. The storage system includes a large number of solid supports having cryopreserved/frozen viable somatic stem and/or progenitor cells from patients, and, preoperative information of the patients from which the cells have been taken. A product which includes viable somatic stem and/or progenitor cells combined with preoperative information of the patient from which the somatic stem and/or progenitor cells have been taken is described. The viable somatic stem and/or progenitor cells stored in the described system are useful in treatment of a disease or a disorder.

### SUBJECT OF THE INVENTION

The present invention relates to a large scale storage system of viable stem and/or progenitor cells of a subject, in particular for later use in a method of treatment of a disease or a disorder, or a predisposition thereof, of said or related subject.

### PRIOR ART AND TECHNOLOGICAL BACKGROUND UNDERLYING THE INVENTION

When stem cells were first isolated, in 1998 it was realized that these were cells which build and repair tissue and blood systems throughout our body, from embryo to adulthood.

In the earliest stages of the embryo they turn into 200 or so types of tissue, from heart cells and red blood cells, to the composition of the eyeball, or liver. It was not long before doctors were implanting them in patients with leukaemia and seeing a whole blood system being created, permanently, where a patient would have previously died. There have been thousands of such cases. Leukemia was the first big success with stem cell transplants but now the field is widening to other illnesses.

when stem cells are available for transplantation to a damaged area, for example in the heart, they are grown in vitro (in a test tube), then transplanted directly at the site of the damage For instance, doctors are targeting stem cells to become islet cells, the cells which make insulin but are missing in the pancreas for those with diabetes. For other applications, said cells may be injected into the bloodstream of the patient.

many articles and patent applications/patents are available describing the generations of high quality stem cell suspensions, their enrichment and their use in medical treatments. Pluripotent stem cells may be isolated from embryonic, foetal and adult tissues. Hematopoietic stem-cell therapy is a clinical reality for almost 40 years, use of stem cells to produce all sorts of replacement parts of the human body is under investigation.

U.S. Pat. No. 4,714,680 discloses cell suspensions comprising human stem and progenitor cells and methods for isolating such suspensions, and the use of the cell suspensions for hematopoietic reconsititution.

EP 0 343 217 and U.S. Pat. No. 5,004,681 relate to the isolation and the reservation of foetal and neonatal hematopoietic stem and progenitor cells of the blood. According to said patents, the use of human neonatal (umbilical cord blood) and foetal blood is preferentially used as this contains much higher levels of said cells than those found in the adult.

U.S. Pat. No. 4,721,096 describes a process for replicating bone marrow in vitro and using the same.

U.S. Pat. No. 5,972,627 and U.S. Pat. No. 5,681,559 describe methods of purifying a population of cells enriched for haematopoietic progenitors or stem cells, respectively.

### SUMMARY OF THE INVENTION

At this moment institutes, labs and/or hospitals have their own (small) storage system for the preservation of isolated stem and/or progenitor cells. Only specialized persons who are working in the field of cell transplantation know how to proceed and who to contact in order to allow the preservation of their own stem and/or progenitor cells which may be used later in a method of treatment of a disease or a disorder, or a predisposition thereof. There is a need to make the use of somatic stem and/or progenitors cells accessible for the man-on-the-street and to make its use more efficiently.

The present invention is directed towards providing a storage system especially a large scale storage system of viable somatic stem and/or progenitor cells for use in a method of treatment of a disease or a disorder of a patient, or a predisposition thereof Said large scale system allows for instance to contact candidate donors, to inform these, about the performed sampling/storage method, to determine if they are an effective candidate for said procedure, to inform the patient about the diseases which may are treated at the moment of the sampling, to inform the patient if other persons may be treated using said cells and, to contact on request of the patient hospitals in order to proceed with a cell transplant for a certain patient. As most of this information is centralized through such a system, the efficiency of the cell sampling, preservation and their use becomes much more efficient and accessible for a normal non-medical person (man-on-the-street). Before the filing of the present application, a system as proposed in the present invention was never described nor suggested before.

The present invention thus suggests to collect and store somatic stem and/or progenitor cells for people who are at risk of a certain disease. As all individuals are at risk, the present invention applies for all living creatures, multicellular animals, in particular humans. It is envisaged that the presently claimed systems, methods and compositions can also be used in the veterinary sector to treat diseased domestic animals of great value to their owners. Assistance can be given in assessing the patient's risk on the basis of the genomic profile in combination with the environmental factors. The collection of stem cells made may be located nearby hospitals where cell transplantation may be applied.

The present invention relates thus to the commercial provision of the possibility to sample and store somatic stem cells from an individual, in order to create the possibility to treat said individual, or a related person, with said stem cells when there is a need for a treatment of a disease or a predisposition thereof. Said sampling may be taken after the birth of the individual (post-natal); however the present invention does not exclude the sampling of stem cells prenatal (foetal tissue) or neonatal (i.e. from the umbilical cord).

A first embodiment of the present invention is directed to a large scale storage system of viable somatic stem and/or progenitor cells or tissue comprising the same for use in a method of treatment of a disease or a disorder of a patient, or having a predisposition thereof, comprising a large number of solid supports comprising cryopreserved/frozen viable somatic stem cells or tissues comprising the same from patients, and, preoperative information of the patients from which the cells have been taken.

According to the present invention said cryopreserved/frozen viable somatic stem and/or progenitor cells (tissue) may be made through a method comprising the steps of: a) isolating or obtaining (pre-natal, neonatal or post-natal) tissue from a patient comprising somatic stem and/or progenitor cells, b) optionally, separating the stem and/or progenitor cells from said tissue, and, c) cryopreserved/freezing the tissue of step a) or the cells of step b) in a solid support such that said tissue or cells remain(s) viable.

Furthermore, the system of the present invention may result in obtaining viable (pre-natal, neonatal or post-natal) somatic stem and/or progenitor cells for use in a method of treatment of a disease or a disorder of a patient, or having a predisposition thereof, comprising the steps of: a) isolating or obtaining (pre-natal, neonatal or post-natal) tissue from a patient comprising somatic stem and/or progenitor cells, b) optionally, separating the stem and/or progenitor cells from said tissue, c) cryopreserved/freezing the tissue of step a) or the cells of step b) in a solid support such that the tissue or cells remain(s) viable, and, d) thawing said tissue or cells.

Furthermore, the present invention relates to a method of treatment of a disease or a disorder of a patient, or a method of treatment of a patient having a predisposition for a disease or disorder, comprising thawing tissue comprising somatic stem and/or progenitor cells or thawing isolated stem and/or progenitor cells from patients obtained by means of a large scale storage system according to the present invention and administering said stem and/or progenitor cells to said patient.

In said system or method the solid support may be marked by a barcode.

Preferentially, the tissue is isolated from remote areas of the body of the patient. More in particular, said tissue may be chosen from the group consisting of bone marrow, blood and fat tissue. Even more specifically, said bone marrow may be isolated from hip bones.

The present invention further suggests that the patient from which the tissue is taken may be an adult.

According to the present invention, said cells or tissue may be further treated using stem cell technologies. In addition, said cells or tissue may be further differentiated. For instance the differentiated cells/tissue may be chosen from the group of neuronal, liver, islet and heart cells/tissue.

A second embodiment of the invention relates to a product comprising a plurality of viable somatic stem and/or progenitor cells combined with preoperative information of the patient from which said somatic stem and/or progenitor cells have been taken. Said product may for instance be a frozen or thawed product.

In said product the somatic stem and/or progenitor cells may carry a stably incorporated heterologous gene sequence for use in the treatment or prevention of the human disease or disorder, or a predisposition thereof, said cells being capable of generating progeny cells which express the heterologous gene sequence.

The present invention further elaborates that the disease or disorder (or predispositions thereof) which may be treated using cells or compositions stored according to the system of the present invention may be chosen from the group consisting of leukemia and related cancers such as lymphoma; damages to heart cells and heart vessels, such as those following acute myocardial infarction (heart attack), congestive heart disease, or other heart ailments for example unstable angina pectoris; brain and spinal cord neurological damage (eg. Parkinson's disease and Alzheimer Disease); stroke, and, diabetes (develop islet cells).

Furthermore, a plurality of viable somatic stem and/or progenitor cells stored through a system according to the present invention, or a product comprising the same thereof, may be used in a method to prepare cell transplants; to prepare bio-engineer organ parts (for instance nerve bundles for spinal cord repair; liver, pancreas and so on); to re-build cartilage following sports injuries, accidents, surgery on joints or arthrosis; to repair tissue for cosmetic or reconstructive surgery; to repair skin from burns and grafts; or, to prepare cells which may be used in gene therapy for treating for instance cancers, Cystic Fibrosis, Huntington Disease, Thalassaemia, and Haemophilia.

Preferably, in the system, the method of treatment or the product according to the present invention said patient is treated with autologous cells.

A third embodiment of the invention provides a method for the preservation of viable postnatal stem and/or progenitor cells for use in a method of treatment of a disease or a disorder of a patient, comprising the steps of: a) isolating post-natal tissue from a patient comprising stem and/or progenitor cells, b) optionally, separating the stem cells and/or progenitor cells from said postnatal tissue, and, c) cryopreserved/freezing the tissue of step a) or the cells of step b) in a solid support such that said tissue or cells remain viable.

A fourth embodiment of the present invention is directed to a method for obtaining postnatal stem and/or progenitor cells for use in a method of treatment of a disease or a disorder of a patient, comprising the steps of: a) isolating postnatal tissue from a patient comprising stem and/or progenitor cells, b) optionally, separating the stern End/or progenitor cells fromrsaid postnatal tissue, c) cryopreserved/freezing the tissue of Step a) or the cells of step b) in a solid support such that the tissue or cells remain(s) viable and, d) thawing said tissue or cells.

In the above-mentioned methods, said solid support may be marked by a barcode.

Furthermore, according to the present invention, said postnatal tissue may isolated from remote areas of the body of the patient. For instance, said postnatal tissue is isolated from the group consisting of bone marrow, blood and fat tissue. Even more prefereritially, said bone marrow is isolated from hip bones.

In the methods of the present invention, the patient from which the postnatal tissue is taken may be an adult.

In the method of the present invention said cells or tissue may be further treated using stem cell technologies. In addition, said cells or tissue may be further differentiated. Examples of said differentiated cells may be chosen from the group of neurons, liver cells, islet cells, heart cells.

A fifth embodiment of the present invention relates to a system of preserved viable post-natal stem or progenitor cells for the use in a method of treatment of a disease or a disorder of a patient, comprising (a) solid support(s) comprising cryopreserved/frozen viable post-natal stem and/or progenitor cells from one or more patients, and, preoperative information of the patient(s) from which the postnatal tissue(s) has/have been taken (eg. description of the medical condition of the patient, check list of the information provided to the patient, signed agreement of the patient (Informed Consent)).

A sixth embodiment of the present invention relates to a product comprising a plurality of viable postnatal stem and/or progenitor cells obtained by a method according to the present invention for use in a method of treatment of a disease or a disorder of a patient.

The present invention further indicates that in the cells of the product according to the present invention a heterologous gene sequence of use in the treatment or prevention of the human disease or disorder may be stably incorporated, said cells being capable of generating progeny cells which express the heterologous gene sequence.

Furthermore, in the method, the system or the product according to the present invention said disease or disorder may be chosen from the group consisting of leukemia and related cancers such as lymphoma; damages to heart cells and heart vessels, following acute myocardial infarction (heart attack), congestive heart disease, or other heart ailments for example unstable angina pectoris; brain and spinal cord neurological damage (eg. Parkinson's disease and Alzheimer Disease); stroke, and diabetes (develop islet cells).

The present invention also contemplates that a product or system comprising a plurality of viable postnatal stem and/or progenitor cells obtained by a method according to the present invention may be used in a method to prepare bio-engineer organ parts (for instance nerve bundles for spinal cord repair, liver, pancreas and so on); to re-build cartilage following sports injuries, accidents, surgery on joints or arthrosis; to repair tissue for cosmetic or reconstructive surgery; to repair skin from burns and grafts; or, to prepare cells which may be used in gene therapy for treating for instance cancers, Cystic Fibrosis, Huntington Disease, Thalassaemia, and Haemophilia.

In the method, the system, or the product according to the present invention said patient may be treated with autologous cells.

### DETAILED DESCRIPTION OF THE INVENTION

As mentioned above, till the filing of the present application, only small scale systems exist which allow the storage of somatic stem and/or progenitor cells. Said systems are located in specialized hospitals or institutes, and are not known for the man-on-the-street. The present invention proposes to set up a commercial large scale storage system containing a large number of somatic stem and/or progenitor cells; preferentially said system contains thousands of samples comprising somatic stem and/or progenitor cells. It is the first time that a large system is proposed allowing the coordination of the storage of these multipotent cells and their use. Through the set up of such a system it becomes clear for a non-medical trained person where to go and what to do to ensure all possible (known and future) treatments which he may need in the near or far future. As said commercial system may be used by hospitals and/or institutions specialized in cell transplantations, it does also guarantee that the medical application of said cells is performed professionally and thus efficiently. In addition, the medical doctors may rely on the professional information stored by said system. Furthermore, no time is lost when the moment of the preparation of the treatment of a patient occurs. This whole system combines an efficient storage of sampled stem and/or progenitor cells with an efficient application of said cells. It is only through this combination that a person may be convinced that future treatments using cell transplants of his body (or relatives) may be ensured.

An embodiment of the present invention, relates to a large scale storage system of viable somatic stem and/or progenitor cells for the use in a method of treatment of a disease or a disorder of a patient, or having a predisposition thereof, comprising a large number of solid supports comprising cryopreserved/frozen viable somatic stem and/or progenitor cells from patients, and, preoperative information of the patients from which the cells have been taken. Said preoperative information may be stored by means of computer programs.

According to the present invention said large scale storage system may comprise samples of thousands of patients. For security reasons, for each sample of a patient, separate vials of stem cells may be stored in at least two different centres. In addition, for each sample of a patient several vials may be stored in order to allow to treat the patients at different times of his life cycle, subsequent to each other or not.

Mature cells derive from and are replaced, on demand, by morphologically recognizable dividing precursor cells from corresponding lineages. The precursor cells derive from more primitive cells and can simplistically be divided into two major subgroups: stem cells and progenitor cells. The definitions of stem cells are operational and depend on functional, rather than morphological criteria. Stem cells have extensive self-renewal or self-maintenance capacity, a necessity since absence or depletion of these cells could result in the complete depletion of one or more cell lineages, events that would lead within a short time to disease and death. Some of the stem cells differentiate upon need, but some stem cells or their daughter cells produce other stem cell to maintain the precious pool of these cells. Thus in addition to maintaining their own kind, pluripotential stem cells are capable of differentiating into several sublines of progenitor cells with more limited self-renewal capacity or no self-renewal capacity. These precursor cells ultimately give rise to the morphologically recognizable precursor cells. The progenitor cells are capable of proliferating and differentiating along one, or more than one, differentiation pathway(s). Stem cells and progenitor make up a very small percentage of the nucleated cells in bone marrow, spleen and blood.

A somatic stem cell is thus multipotent and can make exact copies of itself indefinitely. In addition, a stem cell has the ability to produce specialized cells for various tissues in the body—such as heart muscle, brain tissue, and liver tissue. There are a variety of stem cell types within the human body, including blood stem cells, muscle/bone stem cells, brain stem cells, and liver stem cells.

Scientists are able to maintain stem cells forever, developing them into specialized cell as needed. These different stem cell types may also be considered as a possible target for gene therapy, as modification of these stem cells will ensure enduring generation of progeny containing the corrective gene. In said case corrective DNA may be delivered to said somatic stem cells depending on the disease to be treated. However, unlike germ-line cells, genetic correction of somatic stem cells will not result in passage of the correction to children of treated patients.

There are two basic types of stem cells. Embryonic stem cells are obtained from either aborted foetuses or fertilized eggs that are left over from in vitro fertilization (IVF). They are useful for medical and research purposes because they can produce cells for almost every tissue in the body. Adult stem cells are not as versatile for research purposes because they are specific to certain cell types, such as blood, intestines, skin, and muscle. The term “adult stem cell” may be misleading because both children and adults have them. The present invention focuses mainly on the preservation of viable postnatal stem and/or progenitor cells for use in a method of treatment of a disease or a disorder of a patient. However, the sampling of prenatal and neonatal samples for this purpose is not excluded. The principles and the details for the isolation and preservation of foetal and neonatal hematopoietic stem and progenitor cells of the blood may be found in for instance EP 0 343 217 and U.S. Pat. No. 5,004,681. In the present invention, stem cells derived from the placenta or from the umbilical cord are hereby also referred to as somatic stem or progenitor cells. Adequate cell sources for cell transplants are described in Stocum DL 1998, Wound Repair Regen. 1998 July-August, 6(4): 273-5 and Stocum DL Wound Repair Regen. 2001 November-December, 9(6): 429-42). Donor sources for somatic stem cells are also listed in Gojo and Umezawa 2003, March, 16(1): 23-30.

In the past, preference was given to the use of prenatal or neonatal stem cells for use in cell transplantation as the prospects of success in bone marrow transplantation decline in age. Indeed said number and the functionality of said cells declines in adults compared to younger (i.e. neonatal) stem cells. In adults, stem and progenitor cells are mostly confined to the bone marrow; very few circulate in the blood.

The term somatic stem and/or progenitor cells as used in the present invention depict somatic stem cells as well as their progenitors.

In the system, product or method of the present invention, said somatic stem and/or progenitor cells are preferentially taken from post-natal tissues. For instance, in the system, product or method of the present invention, the patient from which the tissue is taken may be an adult.

The present invention combines the possibility to isolate preferentially postnatal somatic stem and/or progenitor cells and the storage of said cells and their essential medical information. This allows to obtain viable multipotent cells which may be used to treat, at a later stage, preferentially autologous, patients with a disease or a predisposition to a certain disease. A person may thus always decide to store own multipotent cells/tissue(s) which may be used to treat him when necessary. For instance, it is possible at the time a person needs the cells to be treated, the medical condition is not optimal for the sampling of said cells. For instance said person may be infected, exhausted through which said stem or progenitor cells may be of low quality so that they an not be used anymore for the treatment of said person. Furthermore, it is beneficial that postnatal cells may be used for said approach as in most cases no cell samples are taken prenatal. Indeed, it is nearly inconceivable that a mother, before the baby is born, asks for the sampling of stem cells of said baby. Indeed, cell sampling in this case may lead to a natural abortion which is unacceptable. Alternatively, neonatal blood of the umbilical cord and placenta may be taken. An example of which no neonatal samples may be taken is when the delivery is situated at a place where no medical or appropriate medical conditions are present (eg. poor area, or third world countries). Furthermore, it is known that in certain religions the sampling of especially foetal or neonatal cells is prohibited. Therefore it is of great importance that a person may decide at any time of his life to sample and store own stem cells and/or progenitor cells which may be used at later stage for the treatment of his own body or an other body such as a body of a relative.

The idea behind stem cell collection in adults is to store these cells, while those are healthy, and as science moves on utilize them in both current and new procedures. Stem cells need to be taken as early as possible in the adult stage. By getting older, the cells will also lose some of their important capacities. Aging has both quantitative and qualitative effects on stem cells. During normal aging, there is a acutely apparent when subjected to stress; there is a diminished self-renewal capacity, restriction to the breath of developmental potency and a decreased number of progeny of old stem cells. Therefore the present invention suggests, when sampling somatic stem and/or progenitor cells of adults, the age of said adults is, preferentially not higher than 50 to 55 years.

Preoperative information of the patient may include the description of the medical condition of the patient or the medical conditions of family members, a check list of the information provided to the patient, and/or signed agreement of the patient (Informed Consent). All persons from which a sample (will be) is taken are informed how the samples will be taken, stored and how they may be finally used. An Informed Consent is set up. This comprises a clear information in respect of the technique that will be used, the usefulness of the stem/progenitor cells obtained, the eventual use in the future, then risks, the sampling procedure and the storage. Furthermore, the physical condition of the person from which the sample is taken is analyzed before sampling. If the condition does not satisfy the requirements, sampling is not performed.

The volumes of the samples taken may vary, ranging from a couple of μ up to a liter. For instance 500 ml can be sampled; making for example 5 vials of 90 ml each. Each of these tubes may be frozen and thawed separately when needed.

A person skilled in the art knows what type of solid support may be used to store the somatic stem and/or progenitor cells. Said vials are preferentially closed supports such as closed vials of any suitable dimension, shape or material.

Before or after the addition of the sample onto/into said solid support, said support may be marked, allowing a fast and easy identification of the content of the vial. Said mark may be a barcode. In the above-mentioned system, said solid support is preferentially marked by a barcode. As said solid supports may be stored in large storage systems said supports are preferable marked using a system allowing a fast and/or automatic handling of said vials. Equipment which may be used to read said codes are used in other fields and commercial available.

This sampling may be followed by the storage of said samples in a therefore especially designed container allowing the cooling of the sample. Said container is preferentially closed and transported if needed to a lab for further analysis/storage. Both storages may be long term or short term storages.

The freezing of the sample is performed in a controlled way. The viability of bone marrow cells preserved by current methods of cryopreservation exceeds 90%. Examples of systems for freezing bone marrow and biological substances in accordance with a precalculated temperature-time curve are disclosed in U.S. Pat. No. 4,107,937 and U.S. Pat. No. 4,117,881. Preferably, the bone marrow cells are stored in liquid nitrogen at a temperature, e.g. −196° C., at which all activity of the marrow cells, including cell replication, has ceased.

Freezing is destructive to most living cells. Upon cooling, as the external medium freezes, cells equilibrate by losing water, thus increasing intracellular solute concentration. Below about 10-15° C., intracellular freezing will occur. Both intracellular freezing and solution effects are responsible for cell injury (Mazur, P., 1970, Science 168:939-949). It has been proposed that freezing destruction from extracellular ice is essentially a plasma membrane injury resulting from osmotic dehydration of the cell (Meryman, H. T., et al., 1977, Cryobiology 14:287-302).

Cryoprotective agents and optimal cooling rates can protect against cell injury. Cryoprotection by solute addition is thought to occur by two potential mechanisms: colligatively, by penetration into the cell, reducing, the amount of ice formed; or kinetically, by decreasing the rate of water flow out of the cell in response to a decreased vapor pressure of external ice (Meryman, H. T., et al., 1977, Cryobiology 14:287-302). Different optimal cooling rates have been described for different cells. Various groups have looked at the effect of cooling velocity or cryopreservatives upon the survival or transplantation efficiency of frozen bone marrow cells or red blood cells (Lovelock, J. E. and Bishop, M. W. H., 1959, Nature 183:1394-1395; Ashwood-Smith, M. J., 1961, Nature 190:1204-1205; Rowe, A. W. and Rinfret, A. P., 1962, Blood 20:636; Rowe, A. W. and Fellig, J., 1962, Fed. Proc. 21:157; Rowe, A. W., 1966, Cryobiology 3(1):12-18; Lewis, J. P., et al., 1967, Transfusion 7(1):17-32; Rapatz, G., et al., 1968, Cryobiology 5(1):18-25; Mazur, P., 1970, Science 168:939-949; Mazur, P., 1977, Cryobiology 14:251-272; Rowe, A. W. and Lenny, L. L., 1983, Cryobiology 20:717; Stiff, P. J., et al., 1983, Cryobiology 20:17-24; Gorin, N. C., 1986, Clinics in Haematology 15(1):19-48). The successful recovery of human bone marrow cells after long-term storage in liquid nitrogen has been described (1983, American Type Culture Collection, Quarterly Newsletter 3(4):1). In addition, stem cells in bone marrow were shown capable of withstanding cryopreservation and thawing without significant cell death, as demonstrated by the ability to form equal numbers of mixed myeloid-erythroid colonies in vitro both before and after freezing (Fabian, I., et al., 1982, Exp. Hematol. 10(1):119-122). The cryopreservation and thawing of human foetal liver cells (Zuckerman, A. J., et al., 1968, J. Clin. Pathol. (London) 21(1):109-110), foetal myocardial cells (Robinson, D. M. and Simpson, J. F., 1971, In Vitro 6(5):378), neonatal rat heart cells (Alink, G. M., et al., 1976, Cryobiology 13:295-304), and foetal rat pancreases (Kemp, J. A., et al., 1978, Transplantation 26(4):260-264) have also been reported.

In the system of the present invention, the cryopreserved somatic stem and/or progenitor cells or tissue comprising the same may be made through a method comprising the steps of: a) isolating (prenatal, neonatal and postnatal) tissue from a patient comprising somatic stem and/or progenitor cells, b) optionally, separating the stem cells from said tissue, and, c) cryopreserved/freezing the tissue of step a) or the cells of step b) in a solid support such that said tissue or cells remain viable.

Furthermore, said system, may result in obtaining viable (pre-, neo- and postnatal) somatic stem and/or progenitor cells or tissue comprising the same for use in a method of treatment of a disease or a disorder of a patient, or having a predestination thereof, comprising the steps of: a) isolating or obtaining (pre-natal, neonatal or post-natal) tissue from a patient comprising somatic stem and/or progenitor cells, b) optionally, separating the stem and/or progenitor cells from said tissue, c) cryopreserviing/freezing the tissue of step a) or the cells of step b) in a solid support such that the tissue or cells remain(s) viable, and, d) thawing said tissue or cells. According to the present invention, separation of the stem and/or progenitor cells from said tissue may, occur before or after the cryopreservation of said cells.

Said method may further comprise a step of replicating the bone marrow cells in vitro as described in U.S. Pat. No. 4,721,096. In summary, the bone marrow cells retrieved from cryopreservative storage are first separated from their reticulum. The bone marrow cells are then grown in co-cultures with stromal components of normal marrow including fibroblasts, macrophages, reticular cells, and adipocytes or with factors derived from culture media or these cells as well as substances produced in vitro by hepatic (liver) and splenic (spleen) macrophages. Although marrow cells are capable of limited growth when cultured alone, long term growth of these cultures is possible only if stromal cells or their secretory products are added. The present invention seeks to maximize the proliferation of a multipotential hematopoietic stem cell which has the capability of repopulating bone marrow which has been destroyed by intrinsically or environmentally-mediated disease or by the treatment of such disease with chemotherapy and/or radiation. Stem cells which have marrow repopulating activity (MRA) have been shown to persist and replicate in long term bone marrow cultures.

As explained above, many areas of the body may be used as source material for the somatic stem and/or progenitor cells. According to the present invention, said tissue may be isolated from remote areas of the body of the patient. For instance, said tissue is isolated from the group consisting of bone marrow, blood and fat tissue. Preferentially, said bone marrow is isolated from hip bones.

In accordance with the method of the present invention, an appropriate amount of bone marrow may be aspirated of a donor. Methods of aspirating bone marrow from a donor are well known in the art. Examples of apparatus and processes for aspirating bone marrow from a donor can be found in U.S. Pat. No. 4,481,946 and U.S. Pat. No. 4,486,188.

In the system, product or method of the present invention said cells or tissue may be further treated using stem cell technologies. The present invention further indicates that in the cells of the product according to the present invention a heterologous gene sequence of use in the treatment or prevention of the human disease or disorder may be stably incorporated, said cells being capable of generating progeny cells which express the heterologous gene sequence.

Gene therapy refers to the transfer and stable insertion of new genetic information into cells for the therapeutic treatment of diseases or disorders. The foreign gene is transferred into a cell that proliferates to spread the new gene throughout the cell population. Thus stem cells, or pluripotent progenitor cells, are usually the target of gene transfer, since they are proliferative cells that produce various progeny lineages which will potentially express the foreign gene.

Most studies in gene therapy have focused on the use of hematopoietic stem cells. High efficiency gene transfer systems for hematopoietic progenitor cell transformation have been investigated for use (Morrow, J. F., 1976, Ann. N.Y. Acad. Sci. 265:13; Salzar, W., et al., 1981, in Organization and Expression of Globin Genes, A. R. Liss, Inc., New York, p. 313; Bernstein, A., 1985, in Genetic Engineering: Principles and Methods, Plenum Press, New York, p. 235; Dick, J. E., et al., 1986, Trends in Genetics 2:165). Reports on the development of viral vector systems indicate a higher efficiency of transformation than DNA-mediated gene transfer procedures (e.g., CaPO4 precipitation and DEAE dextran) and show the capability of integrating transferred genes stably in a wide variety of cell types. Recombinant retrovirus vectors have been widely used experimentally to transduce hematopoietic stem and progenitor cells. Genes that have been successfully expressed in mice after transfer by retrovirus vectors include human hypoxanthine phosphoribosyl transferase (Miller, A., et al., 1984, Science 255:630). Bacterial genes have also been transferred into mammalian cells, in the form of bacterial drug resistance gene transfers in experimental models. The transformation of hematopoietic progenitor cells to drug resistance by eukaryotic virus vectors, has been accomplished with recombinant retrovirus-based vector systems (Hock, R. A. and Miller, A. D., 1986, Nature 320:275-277; Joyner, A., et al., 1983, Nature 305:556-558; Williams, D. A., et al., 1984, Nature 310:476-480; Dick, J. E., et al., 1985, Cell 42:71-79); Keller, G., et al., 1985, Nature 318:149-154; Eglitis, M., et al., 1985, Science 230:1395-1398). Recently, adeno-associated virus vectors have been used successfully to transduce mammalian cell lines to neomycin resistance (Hermonat, P. L. and Muzyczka, N., 1984, supra; Tratschin, J. D., et al., 1985, Mol. Cell. Biol. 5:3251). Other viral vector systems that have been investigated for use in gene transfer include papovaviruses and vaccinia viruses (see Cline, M. J., 1985, Pharmac. Ther. 29:69-92).

Other methods of gene transfer include microinjection, electroporation, liposomes, chromosome transfer, and transfection techniques (Cline, M. J., 1985, supra). Salser et al. used a calcium-precipitation transfection technique to transfer a methotrexate-resistant dihydrofolate reductase (DHFR) or the herpes simplex virus thymidine kinase gene, and a human globin gene into murine hematopoietic stem cells. In vivo expression of the DHFR and thymidine kinase genes in stem cell progeny was demonstrated (Salser, W., et al., 1981, in Organization and Expression of Globin Genes, Alan R. Liss, Inc., New York, pp. 313-334).

Gene therapy has also been investigated in murine models with the goal of enzyme replacement therapy. Thus, normal stem cells from a donor mouse have been used to reconstitute the hematopoietic cell system of mice lacking beta-glucuronidase (Yatziv, S., et al., 1982, J. Lab. Clin. Med. 90:792-797). Since a native gene was being supplied, no recombinant stem cells (or gene transfer techniques) were necessary.

In addition, in the system, product or method of the present invention the cells or tissue may be further differentiated. Examples of said differentiated cells may be chosen from the group of neurons, liver cells, islet cells, heart cells. Each differentiation requires specific culturing conditions. A skilled person is aware of said specific conditions. Furthermore, said conditions may be optimized or new differentiation conditions may be applied.

According to the present invention the disease or disorder to be treated using the stored cells/tissue or systems or products comprising the same of the present invention may be chosen from the group consisting of leukemia and related cancers such as lymphoma; damages to heart cells and heart vessels, following acute myocardial infarction (heart attack), congestive heart disease, or other heart ailments for example unstable angina pectoris; brain and spinal cord neurological damage (eg. Parkinson's disease and Alzheimer Disease); stroke, and diabetes (develop islet cells).

The present invention also contemplates a plurality of viable somatic stem and/or progenitor cells stored through the system of the present invention, or a product comprising the same, for use in methods to prepare cell transplants; to prepare bio-engineer organ parts (for instance nerve bundles for spinal cord repair, liver, pancreas and so on); to re-build cartilage following sports injuries, accidents, surgery on joints or arthrosis; to repair tissue for cosmetic or reconstructive surgery; to repair skin from burns and grafts; or, to prepare cells which may be used in gene therapy for treating for instance cancers, Cystic Fibrosis, Huntington Disease, Thalassaemia, and Haemophilia. However, as stem cells allow to generate diverse differentiated cells/tissues it is obvious that said cells may be used to treat various disease, disorders or predispositions thereof or may be used in diverse cell therapies. At this moment protocols exist to generate certain differentiated cells/tissues, however in the future the generation of other differentiated cells/tissues will be possible. Therefore, said listing should be interpreted as examples of diseases/disorders or cell therapies but the diseases/disorders to be treated are not limited to said list.

In the system, the method or the product according to the present invention said patient may be treated with autologous cells. With your own body's currently healthy stem cells, there is no possibility of rejection or contamination, as with donor stem cells. However, it is not excluded that other patients may be treated using non-autologous cells. The doctor determines if a patient is may be a good recipient for the introduction of said non-autologous cells.

### DESCRIPTION OF A PREFERRED EMBODIMENT OF THE INVENTION

A preferred embodiment of the present invention is a large scale storage system of viable somatic stem and/or progenitor cells for use in a method of treatment of a disease or a disorder of a patient, or having a predisposition thereof, comprising:
a large number of solid supports comprising cryopreserved/frozen viable somatic stem and/or progenitor cells from patients, and,
preoperative information of the patients from which the cells have been taken, wherein said system comprises: 1/forms needed before the storage of said stem cells:
   forms to contact candidate donors,
   forms to inform the presumed donors about the performed sampling/storage method,
   check list to determine if a presumed donor is an effective candidate for said procedure or not,
   forms to inform the patient about the diseases which may be treated at the moment of the sampling,
   forms to update said listing of diseases which may be treated using stem and/or progenitor cells, and,
   forms to inform the patient if other persons may be treated using said cells, 2/forms needed after the storage of said stem cells, upon request of the patient:
   forms to contact hospitals in order to proceed with a cell transplant for a certain patient, 3/said (pre)operative information comprises
   one or more copies of the forms described above signed by the patient proving his approval (Informed Consent),
   data explaining the medical condition of the patient and his cells before and during the sampling of the stem cells, and,
   approval of a presumed donor as effective candidate for said procedure.

All these forms and information are optional and all of them are only present in the most optimal set up of the system of the present invention. It is possible that certain forms or information are given by other instances (such as hospitals) so that they are not needed in the present system.

In a more preferred embodiment, in said system, the somatic stem and/or progenitor cells are adult bone marrow cells isolated from the hip. Furthermore, the cells obtained via the system of the present invention are preferentially used to treat patients autologously.

Unless other wise defined, all technical and scientific terms used herein have the same meaning as commonly understood by one of ordinary skill in the art to which this invention belongs. Exemplary methods and materials are described below, although methods and materials similar or equivalent to those described herein can be used in the practice or testing of the present invention. All publications and other references mentioned herein are incorporated by reference in their entirety. In case of conflict, the present specification, including definitions, will control. The materials, methods, and examples are illustrative only and not intended to be limiting. Other features and advantages of the invention will be apparent from the following drawings, detailed description, and from the claims.

### MODES FOR CARRYING OUT THE INVENTION

### EXAMPLE 1

### Sampling of the Bone Marrow from the Hip

The procedure is safe, simple, and only requires a local anaesthetic in the hip. The physician administers a local anaesthetic and makes a small ‘nick’ in the skin, in order to insert a fine syringe (needle) through your hip tissue and into the centre of the hip bone (mini-punction). This is one of the largest bones in the body, rich in cell-producing marrow, and a sample of the bone marrow can be drawn up into the syringe. There is no possible harm and a very short recovery period. Within an hour recovery is guaranteed,

### EXAMPLE 2

### Separation of the Stem and/or Progenitor Cells

In ultra-sterile laboratories, the stem cells are separated from the marrow by a separation process.

### EXAMPLE 3

### Freezing of the Stem and/or Progenitor Cells

Subsequently said stem cells are frozen at a controlled rate, so they are available for a decade or more into the future. Preferably 4 separate vials of stem cells in two different centres are kept for security reasons.

### EXAMPLE 4

### Use of Frozen Stem and/or Progenitor Cells

The doctor or surgeon has access to more than one sample of stem cells, at different times, should one need them (or a family member). Said stem cells can be multiplied or modified in the lab before using them as any future cell-transplant.

Its is to be understood that while the invention has been described in conjunction with the detailed description thereof, the foregoing description is intended to illustrate and not limit the scope of the invention, which is defined by the scope of the appended claims. All of the references cited in the description are incorporated by reference. Other aspects, advantages, and modifications are within the scope of the following claims.

1. A large scale storage system of viable somatic stem and/or progenitor cells suitable for use in a method of treatment of a disease or a disorder of a patient, or a method of treatment of a patient having a predisposition for a disease or disorder, comprising:
a) a large number of solid supports comprising viable somatic stem and/or progenitor cells from patients, and,
b) preoperative information of the patients from which the cells have been taken.

2. The system according to claim 1, wherein the cryopreserved/frozen viable somatic stem and/or progenitor cells are made through a method comprising the steps of:
a) isolating or obtaining (pre-natal, neonatal or post-natal) tissue from a patient comprising somatic stem and/or progenitor cells,
b) separating the stem and/or progenitor cells from said tissue, and,
c) cryopreserved/freezing the cells of step b) in a solid support such that said cells remain(s) viable.

3. A method of treatment of a disease or a disorder of a patient, or a method of treatment of a patient having a predisposition for a disease or disorder, comprising thawing tissue comprising somatic stem and/or progenitor cells or thawing isolated stem and/or progenitor cells from patients obtained by means of a large scale storage system of claim 1 and administering said stem and/or progenitor cells to said patient.

4. The system according to claim 1, wherein said solid support is marked by a barcode.

5. The system according to claim 2, wherein said tissue is isolated from remote areas of the body of the patient.

6. The system according to claim 2, wherein said tissue selected from the group consisting of bone marrow, blood and fat tissue.

7. The system according to claim 6, wherein said bone marrow is isolated from hip bones.

8. The system according to claim 2, wherein the patient from which the tissue is taken is an adult.

9. The system according to claim 2, wherein said cells or tissue are/is further treated using stem cell technologies.

10. The method according to claim 3, wherein said cells or tissue are/is further differentiated.

11. The method according to claim 10, wherein the differentiated cells/tissue are/is selected from the group consisting of neuronal, liver, islet and heart cells/tissue.

12. A product comprising a plurality of viable somatic stem and/or progenitor cells combined with preoperative information of a patient from which said somatic stem and/or progenitor cells have been taken.

13. The product according to claim 12, wherein said somatic stem and/or progenitor cells carry a heterologous gene sequence for use in the treatment or prevention of human disease or disorder or a predisposition thereof, said gene sequence being stably incorporated in said cells, said cells being capable of generating progeny cells which express the heterologous gene sequence.

14. The method according to claim 3, wherein said disease or disorder is chosen selected from the group consisting of:
a) leukemia and related cancers such as lymphoma,
b) damages to heart cells and heart vessels, such as those following acute myocardial infarction (heart attack), congestive heart disease, or other heart ailments for example unstable angina pectoris,
c) brain and spinal cord neurological damage (eg. Parkinson's disease and Alzheimer Disease),
d) stroke, and,
e) diabetes (develop islet cells).

15. (canceled)

16. (canceled)

17. (canceled)

18. (canceled)

19. (canceled)

20. (canceled)

21. The method according to claim 3, wherein said patient is treated with autologous cells.

22. A method for the preservation of viable postnatal stem and/or progenitor cells for use in a method of treatment of a disease or a disorder of a patient, comprising the steps of:
a) isolating post-natal tissue from a patient comprising stem and/or progenitor cells,
b) optionally, separating the stem cells and/or progenitor cells from said postnatal tissue, and,
c) cryopreserved/freezing the tissue of step a) or the cells of step b) in a solid support such that said tissue or cells remain(s) viable.

23. The method according to claim 22, further comprising
d) thawing said tissue or cells.

24. The method according to claim 22, wherein said solid support is marked by a barcode.

25. The method according to claim 22, wherein said postnatal tissue is isolated from remote areas of the body of the patient.

26. The method according to claim 22, wherein said postnatal tissue is isolated from bone marrow, blood or fat tissue.

27. The method according to claim 26, wherein said bone marrow is isolated from hip bones.

28. The method according to claim 22, wherein the patient from which the postnatal tissue is taken is an adult.

29. The method according to claim 22, wherein said cells or tissue are/is further treated using stem cell technologies.

30. The method according to claim 23, wherein said cells or tissue are/is further differentiated.

31. A system of preserved viable post-natal stem and/or progenitor cells for the use in a method of treatment of a disease or a disorder of a patient, comprising:
a) (a) solid support(s) comprising cryopreserved/frozen viable post-natal stem and/or progenitor cells from one or more patients, and,
b) preoperative information of the patient(s) from which the postnatal tissue(s) has/have been taken.

32. A product comprising a plurality of viable postnatal stem and/or progenitor cells obtained by a method according to claim 22.

33. The product according to claim 32, wherein said cells carry a heterologous gene sequence, which is stably incorporated in said cells, said cells being capable of generating progeny cells which express the heterologous gene sequence.

34-41. (canceled)
